(19) United States
(12) Patent Application Publication
(10) Pub. No.: US 2014/0323901 A1
(43) Pub. Date: Oct. 30, 2014

(54) ROBOT-BASED AUTISM DIAGNOSIS DEVICE USING ELECTROENCEPHALOGRAM AND METHOD THEREFOR

(71) Applicant: KOREA INSTITUTE OF SCIENCE AND TECHNOLOGY, Seoul (KR)

(72) Inventors: Dong Hwan Kim, Hanam-city (KR); Sung Kee Park, Seoul (KR); Jong Suk Choi, Seoul (KR)

(73) Assignee: KOREA INSTITUTE OF SCIENCE AND TECHNOLOGY, Seoul (KR)

(21) Appl. No.: 14/360,743

(22) PCT Filed: Oct. 22, 2012
(86) PCT No.: PCT/KR2012/008643

§ 371 Date: May 27, 2014

(30) Foreign Application Priority Data
Nov. 25, 2011 (KR) 10-2011-0124037

(51) Int. Cl. A61B 5/0484

(52) U.S. Cl. CPC A61B 5/0484; A61B 5/04847; A61B 5/04842; A61B 5/04845
USPC 600/544

(57) ABSTRACT

The present invention relates to a robot-based autism diagnosis device using an electroencephalogram and to a method therefor. The invention comprises: an electroencephalogram signal detector which is worn on or attached to the head of a child showing signs of autism and detects an electroencephalogram signal generated following an event which is provided; and a robot for diagnosing the degree of autism on receiving the electroencephalogram signal detected by the electroencephalogram signal detector. In the invention, a relatively accurate judgment is made as to whether autism is present, and autism diagnosis is achieved at an early stage such that autistic children can receive treatment for the signs of autism at an early stage.

### TECHNICAL FIELD

The present invention relates to a robot-based autism diagnosis device using electroencephalogram and a method thereof, and more particularly to a robot-based autism diagnosis device using electroencephalogram and a method thereof, in which an early diagnosis of autism is possible through an electroencephalogram pattern observed differently from that of a normal child under a certain event generated by a robot capable of attracting attention of an autistic child who lack communicative skills with others.

### BACKGROUND ART

In general, children act and use language for his/her age as they grow.

However, if children act differently from behaviors for his/her age or lack in language ability, guardians think that children is abnormal but it is difficult to determine whether such symptoms show autism or behavioral development is retarded.

Particularly, only behaviors are not enough to diagnose children, who cannot talk, with autism.

Therefore, guardians visit a hospital or the like institution for autism diagnosis in order to determine whether a child is autistic or his/her behavioral development is less advanced.

However, the autism diagnosis in reality is performed in such a manner that the guardians receives and fills up a questionnaire from the relevant institution or writes the symptoms of the child, and a doctor in charge expresses his/her opinion based on the questionnaire and monitoring of a child's behavior.

As described above, the accuracy of the conventional autism diagnosis is doubtful, and doctors in charge cannot rashly diagnose children under 36 months with autism.

In particular, the reason why children under 36 months are not rashly diagnosed with the autism is because guardians may demand compensation for psychological damages experienced by all the while if it is determined that the children are not autistic but less advanced in development

Therefore, for the reasons of the doubtful accuracy and the compensation for the damage that may be demanded, the conventional autism diagnosis is ambiguous for the doctors in charge to make a diagnosis at an early stage even though the sooner autism treatment starts the better.

Above all, there have been papers like the following prior references, where possibility of classifying electroencephalogram characteristics between normal children and the group of autistic patient's brothers and sisters (i.e., high risk for autism; HRA) that is likely to be diagnosed with autism is experimentally verified through measurement of electroencephalogram with regard to the HRA group. However, there is no patent about a robot-based autism diagnosis device using electroencephalogram for an early autism diagnosis

### DISCLOSURE

### Technical Problem

The present invention is conceived to solve the foregoing problems, and an aspect of the present invention is to provide a robot-based autism diagnosis device using electroencephalogram and a method thereof, in which an early diagnosis of autism is possible through an electroencephalogram pattern observed differently from that of a normal child under a certain event generated by a robot capable of attracting attention of an autistic child who lack communicative skills with others.

### Technical Solution

In accordance with one aspect of the present invention, there is provided a robot-based autism diagnosis device using electroencephalogram, comprising: an electroencephalogram signal detector which is worn on or attached to a head of a child who shows symptoms of autism and detects an electroencephalogram signal generated in accordance with provided events; and a robot which receives the electroencephalogram signal detected by the electroencephalogram signal detector and diagnoses a degree of autism, the robot comprising an event provider which provides an event to the child; an electroencephalogram signal receiver which receives an electroencephalogram signal detected by the electroencephalogram signal detector in accordance with the event provided by the event provider; a controller which analyzes the electroencephalogram signal and diagnoses whether the autism is present and a degree of the autism; and an attention inducer which induces the child to pay attention.

In accordance with one aspect of the present invention, there is provided a robot-based autism diagnosis method using electroencephalogram, comprising: by an event provider under control of a controller, driving an attention inducer of a robot to induce the child, who is tested for autism, to pay attention; by the robot, attaching an electroencephalogram signal detector to a head of a child; by the event provider of the robot, providing an event to the child; by the electroencephalogram signal receiver of the robot, receiving the electroencephalogram signal detected in the electroencephalogram signal detector in accordance with the provided events; by the controller of the robot, analyzing the received electroencephalogram signal; and by the controller, diagnosing whether the autism is present and a degree of the autism, based on the analyzed electroencephalogram signal.

### Advantageous Effects

Embodiments of the present invention provide a robot-based autism diagnosis method using electroencephalogram and method thereof, in which autism is more accurately determined and thus early diagnosis of the autism is possible so that autistic children can receive treatment for the symptoms of the autism at an early stage and thus lead a normal life since then.

### DESCRIPTION OF DRAWINGS

FIG. 1 is a block diagram of to a robot-based autism diagnosis device using electroencephalogram according to an embodiment of the present invention,

FIG. 2 is a block diagram of a robot in the robot-based autism diagnosis device using electroencephalogram according to an embodiment of the present invention,

FIG. 3 is a flowchart of a robot-based autism diagnosis method using electroencephalogram according to an embodiment of the present invention, and

FIG. 4 is a graph showing a degree of autism in the robot-based autism diagnosis method using electroencephalogram according to an embodiment of the present invention.

### BEST MODE

Hereinafter, exemplary embodiments according to the present invention will be described with reference to accompanying drawings. Also, terms and words used in the following description and claims have to be interpreted by not the limited meaning of the typical or dictionary definition, but the meaning and concept corresponding to the technical idea of the present invention on the assumption that the inventor can properly define the concept of the terms in order to describe his/her own invention in the best way.

Accordingly, the disclosure in the specification and the configurations shown in the drawings are just preferred embodiments of the present invention and do not cover all the technical idea of the present invention. Thus, it should be appreciated that such embodiments may be replaced by various equivalents and modifications at a point of time when the present application is filed.

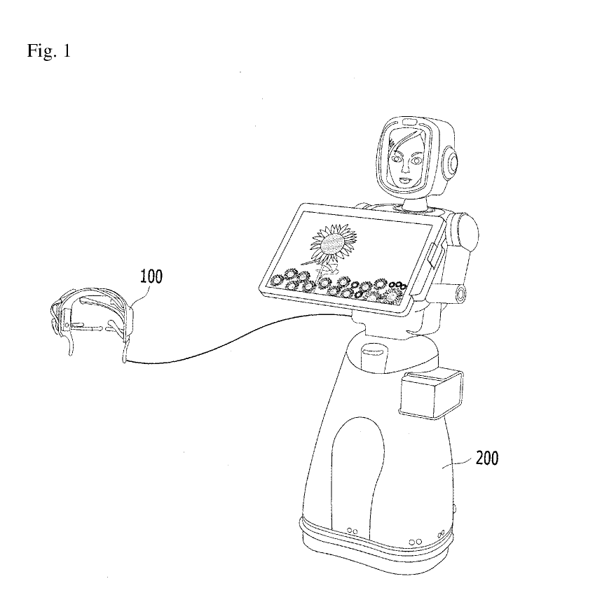

FIG. 1 is a block diagram of to a robot-based autism diagnosis device using electroencephalogram according to an embodiment of the present invention.

As shown in FIG. 1, the robot-based autism diagnosis device using electroencephalogram according to an embodiment of the present invention includes an electroencephalogram signal detector 100 and a robot 200. The electroencephalogram signal detector 100 is worn on or attached to a head of a child who shows symptoms of autism and detects an electroencephalogram signal generated in accordance with provided events, and the robot 200 receives the electroencephalogram signal detected by the electroencephalogram signal detector 100 and diagnoses a degree of autism.

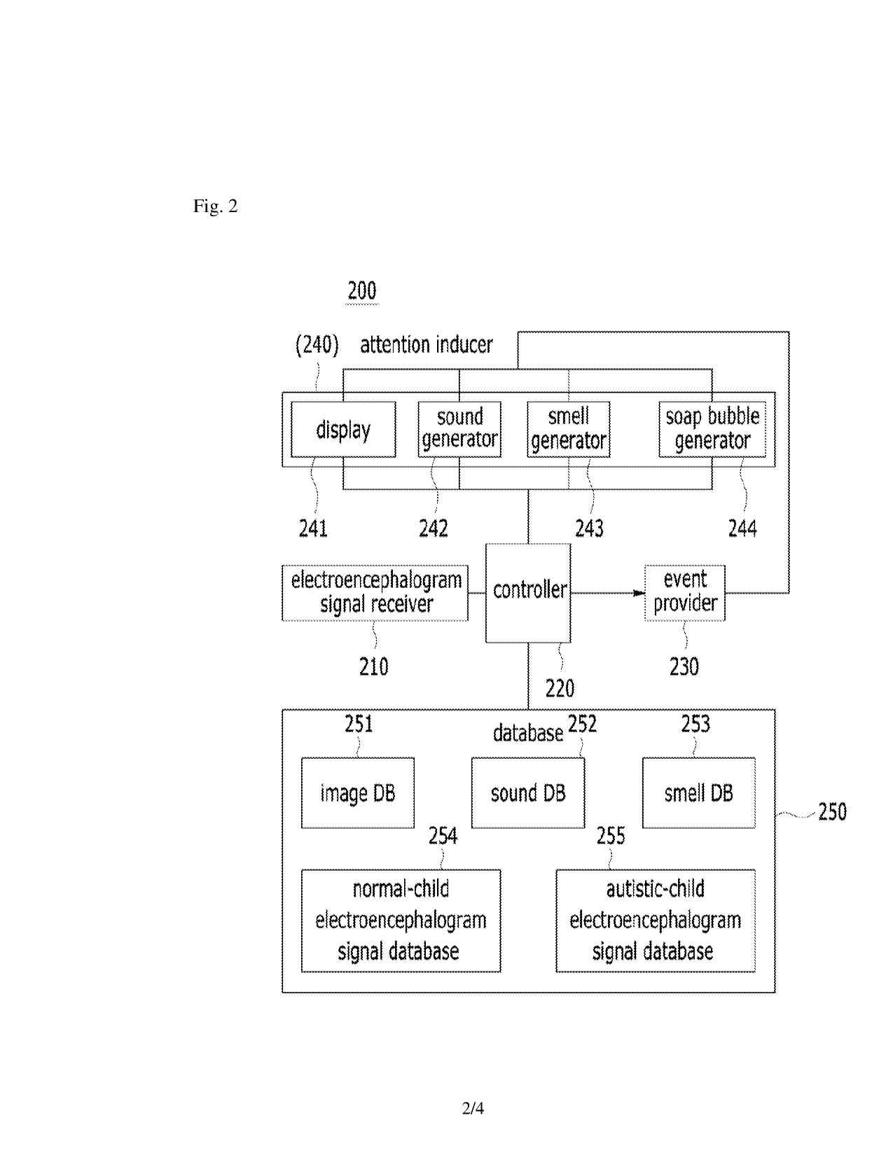

Referring to FIG. 2, the robot 200 will be described in more detail.

FIG. 2 is a block diagram of a robot in the robot-based autism diagnosis device using electroencephalogram according to an embodiment of the present invention.

As shown in FIG. 2, the robot 200 of the robot-based autism diagnosis device using electroencephalogram according to an embodiment of the present invention includes an electroencephalogram signal receiver 210, a controller 220, an event provider 230, an attention inducer 240 and a database 250.

The electroencephalogram signal receiver 210 is connected to the electroencephalogram signal detector 100 through a wire or wirelessly, and receives the electroencephalogram signal detected in accordance with the provided events.

The controller 220 compares the electroencephalogram signal received in the electroencephalogram signal receiver 210 with an electroencephalogram signal of a normal child and analyzes it, thereby diagnosing whether autism is present and a degree of the autism.

Under control of the controller 220, the event provider 230 drives the attention inducer 240 to induce a child who is presumed to be autistic to pay attention, and individually drives a display 241, a sound generator 242, a smell generator 243 and a soap bubble generator 244 involved in the attention inducer 240 to provide events when the electroencephalogram signal detector 100 is attached to the head of the child who is presumed to be autistic.

The database 250 includes an image database 251, a sound database 252, a smell database 253, a normal-child electroencephalogram signal database 254 and an autistic-child electroencephalogram signal database 255.

The image database 251 provides various stored images to the display 241 under control of the event provider 230. The sound generator 242 provides various stored sounds to the sound generator 242 under control of the event provider 230. The smell database 253 provides various stored smells to the smell generator 243 under control of the event provider 230.

The normal-child electroencephalogram signal database 254 provides data so that the controller 220 can compare it with the electroencephalogram signal received from a child who is presumed to be autistic and determine whether s/he is autistic or not. If the child who is presumed to be autistic is diagnosed with autism, the autistic-child electroencephalogram signal database 255 provides data so that the controller 220 can determine a degree of autism.

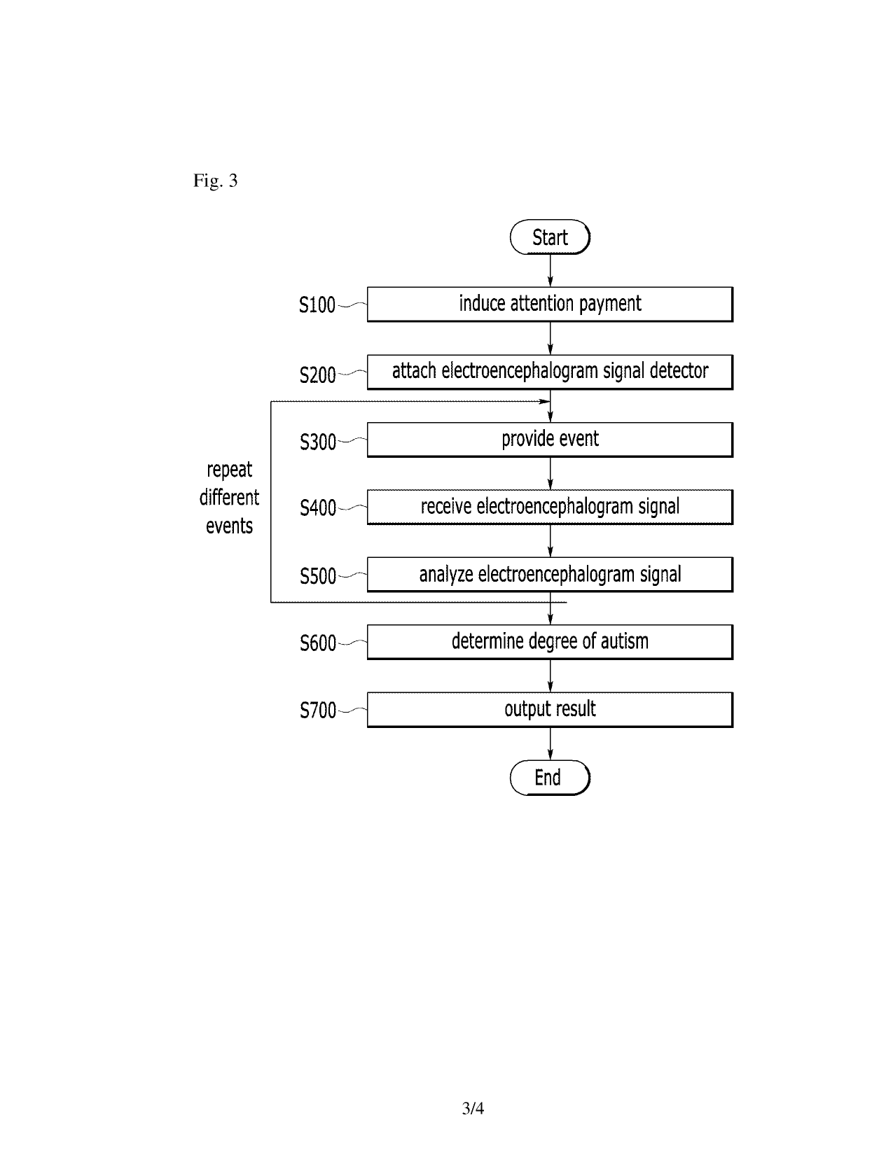

Below, a robot-based autism diagnosis method using electroencephalogram according to an embodiment of the present invention will be described in more detail with reference to FIG. 3.

FIG. 3 is a flowchart of the robot-based autism diagnosis method using the electroencephalogram according to an embodiment of the present invention.

The robot 200 according to an embodiment of the present invention induces a child who is tested for autism to pay attention (S100).

At this time, the induction of the attention payment may be achieved as the event provider 230 of the robot 200 drives the attention inducer 240 to induce a child who is presumed to be autistic to pay attention under control of the controller 220.

In the operation ‘S100’ if a child is induced to pay attention, the robot 200 makes the electroencephalogram signal detector 100 be worn on or attached to the head of the child (S200).

At this time, the electroencephalogram signal detector 100 may be an adhesive thing provided in the form of a sticker, or a wearable thing such as a hat.

The robot 200 provides an event to a child who attaches or wears the electroencephalogram signal detector 100 (S300).

Detailed descriptions about the operation of providing the events are as follows. When the controller 220 controls the event provider 230 to provide the events, the event provider 230 gives an image, a sound and a smell respectively stored in the image database 251, the sound database 252 and the smell database 253 of the database 250 to a user who is attached with the electroencephalogram signal detector 100 through the display 241, the sound generator 242 and the smell generator 243, thereby providing the events.

For instance, the event provider 230 provides a visual event, an auditory event, and an olfactory event through the display 241, the sound generator 242 and the smell generator 243, and additionally provides a tactile event to the child by driving the soap bubble generator 244 to generate soap bubbles.

Let the visual event be A, the auditory event be B, the olfactory event be C, and the tactile event be D. Each event has a plurality of sub events, and let the respective events be {a₁, a₂, . . . aₙ}, {b₁, b₂, . . . bₙ} {c₁, c₂, . . . cₙ} and {d₁, d₂, . . . dₙ}.

That is, the event provider 230 provides a plurality of images, i.e., an image a₁, an image a₂ and an image aₙ stored in the image database 251 to the display 241 in the case of providing the visual event.

At this time, the image a₁, the image a₂ and the image aₙ are different in contents from one another. For example, the image a₁ is an image for a child, the image a₂ is an image of a documentary such as Animal Kingdom, and the image aₙ is an image for advertisement.

The electroencephalogram signal receiver 210 receives an electroencephalogram signal, which is generated in the head of the child as s/he feels or responds to the provided visual event, from the electroencephalogram signal detector 100 (S400).

Of course, the electroencephalogram signal receiver 210 receives the electroencephalogram signal with regard to the auditory event, olfactory event or tactile event after completely receiving the electroencephalogram signal with regard to the visual event.

That is, the electroencephalogram signal receiver 210 always receives the electroencephalogram signal generated in the head of the child who is given the plurality of main events and sub events of each main event whenever each event is provided.

The event provider 230 may provide the visual event, auditory event, olfactory event and the tactile event separately or provide them while mixing the sub events of each event.

The controller 220 analyzes the electroencephalogram signal with regard to each visual event {a1, a2, . . . an} received in the electroencephalogram signal receiver 210 (S500).

For example, the controller 200 extracts a brainwave numerical value x₁ needed for an autism diagnosis from the electroencephalogram signal of the child who watched the image a₁, extracts a brainwave numerical value x₂ for an autism diagnosis with regard to the image a₂, and extracts a brainwave numerical value xₙ for an autism diagnosis with regard to the image aₙ, thereby analyzing the electroencephalogram signal with regard to the plurality of visual events.

Then, the controller 200 diagnoses a degree of autism (S600).

That is, the controller 200 diagnoses the degree of the autism, based on the brainwave numerical values x1, x2, . . . , xn, which are extracted in analyzing the electroencephalogram signal and needed for diagnosing the autism, and based on a autism diagnosis function of data stored in the normal-child electroencephalogram signal database 254 and the autistic-child electroencephalogram signal database 255.

F{x₁, x₂, x₃, x₄ . . . xₙ₋₁, xₙ}>K [Expression 1]

where, F{x ₁, x₂, x₃, x₄, . . . xₙ₋₁, xₙ} is the autism diagnosis function, and K is a threshold for diagnosing whether the autism is present and determining the degree of the autism.

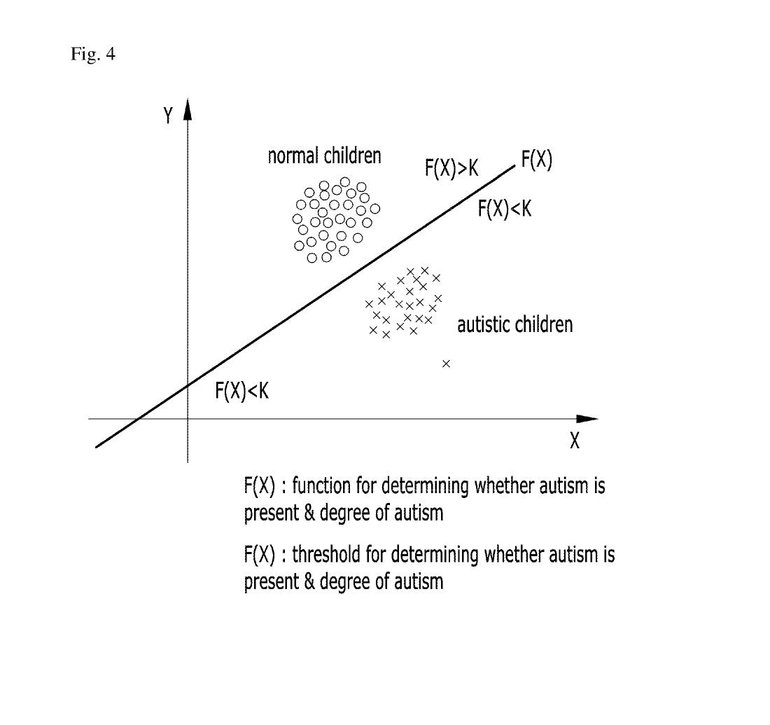

Referring to FIG. 4, it will be described in more detail.

FIG. 4 is a graph showing a degree of autism in the robot-based autism diagnosis method using electroencephalogram according to an embodiment of the present invention.

The controller 220 extracts the brainwave numerical values x₁, x₂. . . xₙ needed for diagnosing the autism from an electroencephalogram signal of a child with respect to the plurality of visual events {a₁, a₂, . . . aₙ}, and enters the brainwave numerical values (x₁, x₂ . . . xₙ) into the autism diagnosis function (F(X)) that has the brainwave numerical value as a variable, thereby diagnosing the child with normality if the calculated value is higher than a threshold (K), but diagnosing the child with autism if the calculated value is smaller than a threshold (K).

At this time, if the value obtained by entering the brainwave numerical values (x₁, x₂ . . . xₙ) into the autism diagnosis function (F(X)) is significantly lower than the threshold, the controller 220 determines that the autism is severe.

That is, as shown in FIG. 4, a normal child and an autistic child respectively form certain distributions with respect to the autism diagnosis function. However, a child may be diagnosed with the severe autism even when the distribution is far away from the autism diagnosis function.

Meanwhile, the controller 220 extracts the brainwave numerical values (y₁, y₂ . . . yₙ) needed for the autism diagnosis from the electroencephalogram signal of the child with regard to the plurality of auditory events {b₁, b₂, . . . bₙ}, and enters the brainwave numerical values (y₁, y₂ . . . yₙ) into the autism diagnosis function ((F(Y)) that has the brainwave numerical value as a variable, diagnosing the child with normality if the calculated value is higher than the threshold, but otherwise diagnosing the child with autism.

Besides the autism diagnosis using the visual event and the auditory event, the autism diagnosis using the olfactory event or the tactile event may also be performed. However, the technical procedures thereof, that is, the procedures of providing the olfactory event or the tactile event and receiving and analyzing the electroencephalogram signal from the child to determine the autism are the same. Therefore, repetitive descriptions thereof will be avoided.

Then, the controller 220 output a diagnosis result through a doctor's terminal connected to the display 241 or the robot 200 (S700).

Further, the controller 220 controls the autistic-child electroencephalogram signal database 255 to store the electroencephalogram signal received by the electroencephalogram signal receiver 210, the analysis result from analyzing the electroencephalogram signal, and the diagnosis about whether the autism is present and the degree of the autism through the analyzed electroencephalogram signal, and continuously accumulates the data to be utilized as more clear data in testing for autism of children since then.

Although some embodiments have been described herein with reference to the accompanying drawings, it will be understood by those skilled in the art that these embodiments are provided for illustration only, and various modifications, changes, alterations and equivalent embodiments can be made without departing from the scope of the present invention.

1. A robot-based autism diagnosis device using electroencephalogram, comprising:
an electroencephalogram signal detector which is worn on or attached to a head of a child who shows symptoms of autism and detects an electroencephalogram signal generated in accordance with provided events; and
a robot which receives the electroencephalogram signal detected by the electroencephalogram signal detector and diagnoses a degree of autism.

2. The robot-based autism diagnosis device using electroencephalogram according to claim 1, wherein the robot comprises:
an event provider which provides an event to the child;
an electroencephalogram signal receiver which receives an electroencephalogram signal detected by the electroencephalogram signal detector in accordance with the event provided by the event provider; and
a controller which analyzes the electroencephalogram signal and diagnoses whether the autism is present and a degree of the autism.

3. The robot-based autism diagnosis device using electroencephalogram according to claim 2, wherein the robot further comprises an attention inducer which induces the child to pay attention.

4. The robot-based autism diagnosis device using electroencephalogram according to claim 3, wherein the event provider provides various images stored in an image database as a plurality of visual events through a display of the attention inducer.

5. The robot-based autism diagnosis device using electroencephalogram according to claim 3, wherein the event provider provides various sounds stored in a sound database as a plurality of auditory events through a sound generator of the attention inducer.

6. The robot-based autism diagnosis device using electroencephalogram according to claim 3, wherein the event provider provides various smells stored in a smell database as a plurality of olfactory events through a smell generator of the attention inducer.

7. The robot-based autism diagnosis device using electroencephalogram according to claim 3, wherein the event provider provides a plurality of tactile events through a soap bubble generator of the attention inducer.

8. The robot-based autism diagnosis device using electroencephalogram according to claim 4, wherein the controller analyzes the electroencephalogram signal by extracting a brainwave numerical value needed for diagnosing the autism from the electroencephalogram signals generated in accordance with the plurality of provided events.

9. The robot-based autism diagnosis device using electroencephalogram according to claim 8, wherein the controller diagnoses whether the autism is present and a degree of the autism, based on whether a value obtained by entering a brainwave numerical value extracted in analyzing the electroencephalogram signal into an autism diagnosis function having the brainwave numerical value as a variable is equal to or higher or lower than a threshold.

10. A robot-based autism diagnosis method using electroencephalogram, comprising:
(B) by a robot, attaching an electroencephalogram signal detector to a head of a child;
(C) by an event provider of the robot, providing an event to the child;
(D) by the electroencephalogram signal receiver of the robot, receiving the electroencephalogram signal detected in the electroencephalogram signal detector in accordance with the provided events;
(E) by the controller of the robot, analyzing the received electroencephalogram signal; and
(F) by the controller, diagnosing whether the autism is present and a degree of the autism, based on the analyzed electroencephalogram signal.

11. The robot-based autism diagnosis method using electroencephalogram according to claim 10, further comprising: (A) by the event provider under control of the controller, driving an attention inducer of the robot to induce the child, who is tested for autism, to pay attention, before (B).

12. The robot-based autism diagnosis method using electroencephalogram according to claim 11, wherein the event provider provides various images stored in an image database of a database as a plurality of visual events through a display of the attention inducer.

13. The robot-based autism diagnosis method using electroencephalogram according to claim 11, wherein the event provider provides various sounds stored in a sound DB of a database as a plurality of auditory events through a sound generator of the attention inducer.

14. The robot-based autism diagnosis method using electroencephalogram according to claim 11, wherein the event provider provides various smells stored in a smell DB of a database as a plurality of olfactory events through a smell generator of the attention inducer.

15. The robot-based autism diagnosis method using electroencephalogram according to claim 11, wherein the event provider provides a plurality of tactile events through a soap bubble generator of the attention inducer.

16. The robot-based autism diagnosis method using electroencephalogram according to claim 12, wherein the analysis of the electroencephalogram signal comprises extracting a brainwave numerical value needed for diagnosing the autism from the electroencephalogram signals generated in accordance with the plurality of provided events.

17. The robot-based autism diagnosis method using electroencephalogram according to claim 16, wherein the controller diagnoses whether the autism is present and a degree of the autism, based on whether a value obtained by entering a brainwave numerical value extracted in analyzing the electroencephalogram signal into an autism diagnosis function having the brainwave numerical value as a variable is equal to or higher or lower than a threshold.
